(19) United States
(12) Patent Application Publication
(10) Pub. No.: US 2026/0218248 A1
(43) Pub. Date: Jul. 30, 2026

(54) INCREASING THE AVAILABILITY OF FERMENTABLE SUGARS IN FERMENTATIONS

(71) Applicant: Danisco US Inc., Palo Alto, CA (US)

(72) Inventors: Bart C Koops, Pijnacker (NL); Nadia Ramirez-Angulo, Leiden (NL); Sindy Morales-Suan, Voorburg (NL); Jan Hendrik A Van Tuijl, Zoetermeer (NL)

(21) Appl. No.: 19/144,969

(22) PCT Filed: Jan. 3, 2024
(86) PCT No.: PCT/US2024/010154

§ 371 Date: Jul. 1, 2025

Related U.S. Application Data
(60) Provisional application No. 63/437,556, filed on Jan. 6, 2023.

(51) Int. Cl. C12P 7/10; C12N 1/16; C12N 1/20; C12N 9/24; C12P 7/06; C12P 7/56; C12P 13/04; C12P 19/02; C12P 19/14; C12R 1/01; C12R 1/645

(52) U.S. Cl. CPC C12P 7/10; C12N 1/16; C12N 1/20; C12N 9/2402; C12P 7/065; C12P 7/56; C12P 13/04; C12P 19/02; C12P 19/14; C12Y 302/01003; C12Y 302/0102; C12P 2203/00; C12R 2001/01; C12R 2001/645

(57) ABSTRACT

Described is a method for increasing the amounts of fermentable sugars in fermentation substrates by treatment with a combination of an enzyme having transglucosidase activity and an enzyme having glucoamylase activity to hydrolyze oligo and/or polysaccharides that are not conventionally hydrolyzed by glucoamylase alone during fermentation. The method is most effective using fermentation substrates containing low amounts of maltose and maltotriose.

### CROSS REFERENCE TO RELATED APPLICATIONS

This application claims priority to U.S. Provisional Patent Application No. 63/437,556, filed Jan. 6, 2023, the disclosure of which is herein incorporated by reference in its entirety.

### TECHNICAL FIELD

The method relates to increasing the amounts of fermentable sugars in fermentation substrates by treatment with a combination of an enzyme having transglucosidase activity and an enzyme having glucoamylase activity to hydrolyze oligo and/or polysaccharides that are not conventionally hydrolyzed by glucoamylase alone during fermentation. The method is most effective using fermentation substrates containing low amounts of maltose and maltotriose.

### BACKGROUND

Fermentations are often limited by glucose as a primary carbon source for fermenting organisms. Examples of such fermentations include ethanol production by yeast, lactic acid production by lactic acid bacteria and amino acid production by organisms such as Corynebacterium. In each case, final product yield is driven by available glucose. This glucose may be the product of starch hydrolyzing enzymes on suitable starch-containing fermentation substates. Such enzymes hydrolase the majority of the starch, but also produce oligosaccharides that cannot be utilized by commercially relevant fermenting micro-organisms.

The need exists for ways to reduce the production of unfermentable oligosaccharides and increase fermentable sugars in fermentation substrates.

### SUMMARY

Described is an enzymatic method for increasing the amounts of fermentable sugars in fermentation substrates by treatment with a combination of an enzyme having transglucosidase activity and an enzyme having glucoamylase activity to hydrolyze oligo and/or polysaccharides that are not conventionally hydrolysed by an enzyme having only glucoamylase activity during fermentation. Aspects and embodiments of the methods are described in the following, independently-numbered paragraphs.
1. In a first aspect, method for increasing the amount of glucose available for fermentation in a fermentation substrate is provided, comprising; contacting a fermentation substrate with an enzyme having transglucosidase activity and an enzyme having glucoamylase activity at a time when the combined amount of maltose and maltotriose in the fermentation substrate is below a selected amount.
2. In some embodiments of the method of paragraph 1, the selected amount is below 0.5%, below 0.05% and preferably below 0.01% w/v.
3. In some embodiments of the method of paragraph 1 or 2, the fermentation substrate following the contacting with the enzyme having transglucosidase activity has reduced levels of isomaltose, panose and/or maltulose, compared to otherwise identical fermentation substrate not contacted with the enzyme having transglucosidase activity.
4. In some embodiments of the method of any of the preceding paragraphs, the fermentation substrate following contacting with the enzyme having transglucosidase activity does not have a significant increase in amounts of ethyl-glucoside, compared to otherwise identical fermentation substrate not contacted with the enzyme having transglucosidase activity.
5. In some embodiments of the method of any of paragraphs 1-4, the fermentation substrate is contacted with the enzyme having transglucosidase activity and the enzyme having glucoamylase activity simultaneously.
6. In some embodiments of the method of any of paragraphs 1-4, the fermentation substrate is contacted with the enzyme having transglucosidase activity and the enzyme having glucoamylase activity sequentially.
7. In some embodiments of the method of any of the preceding paragraphs, the enzyme having transglucosidase activity is added after the start of fermentation.
8. In some embodiments of the method of any of the preceding paragraphs, the transglucosidase is added 32 hours, 40 hours, 48 hours, 54 hours or 64 hours after the start of fermentation.
9. In some embodiments of the method of any of the paragraphs 1-8, the fermentation substrate is a rich-glucose fermentation substrate.
10. In some embodiments of the method of any of paragraphs 1-8, the fermentation substrate is thin stillage added as backset to a liquefaction.
11. In some embodiments of the method of any of paragraphs 1-8, the fermentation substrate is whole stillage, of which at least a portion is added to a liquefaction.
12. In some embodiments of the method of any of paragraphs 1-8, the fermentation substrate is a starch liquefact.
13. In some embodiments, the method of any of the preceding paragraph, further comprises fermenting the glucose made available for fermentation into ethanol, lactic acid or amino acids.
14. In some embodiments, the method of any of the preceding paragraphs further comprises fermenting the glucose made available for fermentation using yeast and/or bacteria and a fermenting organism.
15. In some embodiments of the method of any of the preceding paragraphs, the amount of enzyme having transglucosidase activity contacting the fermentation substrate is at least 0.017, at least 0.085, at least 0.170, at least 0.850 or at least 1.70 transglucosidase Units per gram substrate dissolved solids.

These and other aspects and embodiments of present methods will be apparent from the description and any accompanying drawings.

### DETAILED DESCRIPTION

### I. Definitions and Abbreviations

Prior to describing the present methods in detail, the following terms are defined for clarity. Terms not defined should be accorded their ordinary meanings as used in the relevant art

As used herein the term “starch” refers to any material comprised of the complex polysaccharide carbohydrates of plants, comprised of amylose and/or amylopectin with the formula (C₆H₁₀O₅)ₓ, wherein X can be any number. In particular, the term refers to any plant-based material including but not limited to grains, grasses, tubers and roots and more specifically wheat, barley, corn, rye, rice, sorghum, legumes, cassava, millet, potato, sweet potato, and tapioca. After purification of the complex polysaccharide carbohydrates from the other plant components, it is called “refined starch.”

As used herein, “maltodextrins” refer to oligosaccharides that are generally produced from starch by partial chemical or enzymatic hydrolysis. The size of the polysaccharides generally ranges from DP3 to DP20 but can be longer.

As used herein, the phrase “degree of polymerization” (DP) refers to the number (n) of anhydroglucopyranose units in a given saccharide. An example of DP1 is the monosaccharides glucose. Examples of DP2 are the disaccharides maltose and isomaltose.

As used herein, the expression “end of fermentation,” abbreviated “EoF,” refers to the stage of fermentation when the economic advantage of continuing fermentation to produce a small amount of additional products is exceeded by the cost of continuing fermentation in terms of fixed and variable costs. In a more general sense, “end of fermentation” refers to the point where a fermentation will no longer produce a significant amount of additional product, i.e., no more than about 1% additional product.

As used herein the term “rich-glucose,” with respect to a fermentable substrate, refers to a fermentation substrate having more than 70% of the sugar content being glucose.

As used herein the term “readily/easily utilized/hydrolysed substrate” refers to substrates that are preferred as donor molecule by TGL in a transglucosidase and/or hydrolysis reaction. The small linear substrates maltose and maltotriose are preferred donor molecules for TGL and are utilized faster than other donor molecules.

The term “dry solids content” (DS) refers to the total solids of a slurry in a dry weight percent basis.

The term “slurry” refers to an aqueous mixture containing insoluble solids.

The phrase “simultaneous saccharification and fermentation (SSF)” refers to a process in the production of biochemicals in which a microbial organism, such as an ethanologenic microorganism, and at least one enzyme, such as an amylase, are present during the same process step.

An “ethanologenic microorganism” refers to a microorganism with the ability to convert a sugar or oligosaccharide to ethanol.

As used herein, a “starch processing enzyme” is an enzyme that depolymerizes a starch substrate (including maltodextrin). Exemplary starch processing enzymes are α-amylase, glucoamylase, β-amylase, pullulanase, α-glucosidase and transglucosidase.

As used herein, “contacting” an enzyme with a substrate refers to bringing the enzyme and substrate together in a common aqueous environment, typically accompanied by mixing to achieve uniform distribution. The term “contacted” is used interchangeably with “treated.”

As used herein, “generating” refers to producing a reaction product as the result of an enzymatic process, and is synonymous with the term, “producing.”

As used herein, “whole stillage” is a product of an ethanol production facility following distillation.

As used herein, “thin stillage” is the liquid portion of whole stillage following separation of solid materials. As used herein, “thin stillage” is also referred to simply as “stillage,” as well as “backset” and “recycle.”

As used herein, “distillers’ grains (DG)” is the solid/slurry component of whole stillage.

As used herein, “distillers’ dried grains (DDG) is DG that have been dried.

As used herein, “distillers’ dried grains with solutes (DDGS) is DG that has been dried along with concentrated thin stillage for added nutritional value.

A used herein, the term “performance benefit” refers to an improvement in a desirable biochemical property of a fermentation substrate, or product obtainable, therefrom, including an improvement that results in an increased amount of fermentable sugars available to fermenting organisms.

As used herein, the singular articles “a,” “an” and “the” encompass the plural referents unless the context clearly dictates otherwise. All references cited herein are hereby incorporated by reference in their entirety.

The following abbreviations/acronyms have the following meanings unless otherwise specified. Not all abbreviations may be used.
° C. degrees Centigrade
DP degree of polymerization
DP3+ DP3 or longer
DPn DP with unknown value
DS dry solids
EoF end of fermentation
g gram
HPAE high-performance anion-exchange chromatography
HPLC high performance liquid chromatography
hr hour
kg kilogram
min minute
ml milliliter
mm millimeter
MT metric ton
NaAc sodium acetate
NaOH sodium hydroxide
nC nano-coulomb
nC·min nano-coulomb minutes
PAD pulsed amperometric detection
PU pullulanase
rpm revolutions per minute
TG or TGL transglucosidase
U or u unit
w/V weight/volume

### II. Enzymes with Transglucosidase Activity for Increasing Fermentable Sugars

### A. Introduction

It has been determined that the presence of an enzyme having transglucosidase (TGL) activity, along with an enzyme having glucoamylase (GA) activity, under selected fermentation conditions, makes additional glucose available to fermenting organisms. This additional glucose represents carbon that would, under the best of circumstances, have previously been lost in the form of unfermentable oligosaccharides. During fermentation, enzymes with TGL activity appear to hydrolyze oligo- and/or polysaccharides that are not hydrolysed by enzymes having GA activity, alone, thereby liberating additional glucose substrate for utilization by fermentation organisms. Notably, in addition to transferring glucose units, TGL can also hydrolyse substrates, especially when the concentration of acceptors other than water is low.

### B. Relevant Enzyme Activities

An enzyme having TGL activity, herein primarily referred to as a transglucosidase (TGL), transfers glucosyl groups from donor molecules to acceptor molecules, in most cases resulting in α1-6 linkages. TGL also hydrolyzes linear and branched maltooligosaccharides into smaller maltooligosaccharides and glucose, which are readily utilized by fermenting organisms. When acceptor oligosaccharide concentrations are low the hydrolysis reaction (transfer to water) is favored over the transfer reaction to organic, hydroxyl-containing donor molecules. Exemplary enzymes are described herein.

Preferred substrates for TGL are those most easily utilized as donor molecules. Short linear maltooligosaccharides are especially good donor molecules. Maltose and maltotriose are the most abundant TGL substrates in starch-based fermentations, and their amounts over the course of fermentation represent a good proxy for the total amounts of TGL substrates that can be used as donor molecules by TGL. Branched maltooligosaccharides are less preferred donor molecules for TGL compared to linear maltooligosaccharide.

TGL products depend on the population of acceptor molecules available. Any molecule with a hydroxyl group, including water, alcohol and most (if not essentially all) oligosaccharides present in a fermentation substrate can be acceptor molecules.

### C. Timing of Addition of Enzymes

The present methods are based in part on the observation that the timing of addition of TGL in fermentation is critical to increasing the production of a desired product. Time of addition during fermentation determines the population of substrate donor molecules available upon TGL addition.

Early in fermentation there are significant amounts of linear maltooligosaccharides present, which are readily hydrolysed by GA into suitable donor molecules for enzymes with TGL activity. Early in fermentation there is also an abundance of potential acceptor molecules, including glucose, maltose, maltotriose, linear and branched oligosaccharides and myriad other molecules. As noted, above, any molecule with a hydroxyl group can potentially be an acceptor, including water and alcohol.

In such a donor and acceptor-rich environment, transglycosylation produces some products that can easily be hydrolysed to glucose by GA (or TGL) and later fermented to a product of interest. However, other TGL products are not good GA substrates, and cannot easily be hydrolyzed by GA into fermentable sugars. For example, branched maltooligosaccharides (whether present at the beginning of fermentation or produced following addition of TGL) are much less preferred GA substrates compared to linear maltooligosaccharides. As a consequence, TGL activity can actually convert substrates that are easy to hydrolyze by GA into substrates that are much more difficult to hydrolyze. Indeed, TGL addition early in fermentation has a negative effect on product yield, resulting in more DPn, and in some cases, more DP3 and DP2.

TGL can hydrolyze some of the branched oligosaccharides that GA cannot easily hydrolyze, holding the promise for some benefit to adding TGL early in fermentation. However, the negative effects generally outweigh the positive effects.

Late in fermentation, including the end of fermentation (EoF), there are fewer easy donor and acceptor molecules overall, which include mainly branched oligosaccharides. When TGL is added at later times, it can hydrolyze some branched molecules that GA cannot hydrolyze, and thereby produce more fermentable glucose that can be converted to valuable products.

### III. Exemplary Enzymes for Use in the Method

### A. Enzymes with Transglucosidase Activity

Preferred enzymes with transglycosylation activity readily utilize short linear oligosaccharides as donors, and transfer a glucose from such donors to acceptors, which can be any molecule with a hydroxyl group, including water, alcohol and any oligosaccharide present in a fermentation substrate.

Enzymes with transglycosylation activity include, but are not limited to, enzymes that have previously been described as or annotated transglucosidases, as well as enzymes having previously unknown activities, or side activities, characteristic of transglucosidases. Transglucosidases (TGL), also known as α-glucosidases and α-D-glucoside glucohydrolases, are enzymes classified as EC 3.2.1.20, and have been identified in numerous organisms. GenBank includes over 400 entries for transglucosidases.

The enzyme exemplified herein is from Aspergillis niger and is expressed in Trichoderma reesei. The enzyme expresses at high levels but is otherwise not recognized as having unique properties compared to other transglucosidases studied. Accordingly, a large number of transglucosidases, derived from many organisms, are believed to be suitable for use in the present methods.

Transglucosidase is preferably added when the combined maltose and maltotriose concentration is below about 0.5% w/v. This can at the initiation of fermentation, or about 10, 20, 30, 40, 50 or 60 hours of fermentation, depending on the fermentation substrate. The TGL dose needed is dependent on the remaining fermentation time and can be between abut 0.01 and 1.0 kg/MT DS.

The exemplified enzyme is commercially available as TRANSGLUCOSIDASE L2000® (IFF) with an activity of 1,700 transglucosidase units (TGU)/g. One TGU is defined as the amount of enzyme required to produce one micromole of panose per minute under the conditions of the assay. Typically, at least of 0.1 kg/MT of TRANSGLUCOSIDASE L2000®/MT of DS is used. In all the work described herein, 0.01 to 1.0 kg/MT DS was used. Exemplary amounts of transglucosidase are about 0.017 to 1.70 TGU/g DS, e.g., about 0.017, 0.085, 0.170, 0.850 and 1.70 TGU/g DS.

While time of addition and dosage are important to the present methods, most important is the relative amount of short linear maltooligosaccharides, which are especially good donor molecules for TGL. Preferably, at the time of addition of TGL, a fermentation substrate contains a combined amount of maltose and maltotriose in the fermentation substrate is below 0.5%, below 0.05% and even below 0.01% w/v. In some embodiments, the fermentation substrate contains below 0.5%, below 0.05% and even below 0.01% w/v maltose. In some embodiments, the fermentation substrate contains below 0.5%, below 0.05% and even below 0.01% w/v maltotriose.

### B. Enzymes with Glucoamylase Activity

Preferred enzymes with glucoamylase activity include, but are not limited to, those enzymes that have previously been described as or annotated “glucoamylase,” as well as enzymes having previously unknown activities, or side activities, characteristic of glucoamylases.

Glucoamylases are enzymes are classified as EC 3.2.1.3, and have been identified in numerous organisms. GenBank includes well over 2,000 entries for glucoamylases. An exemplary glucoamylase is Trichoderma reesei glucoamylase (TrGA), and variants thereof that possess superior specific activity and thermal stability. See, e.g., U.S. Published Application Nos. 2006/0094080, 2007/0004018, and 2007/0015266 (Danisco US Inc.).

Alternatively, the glucoamylase may be from, e.g., Aspergillus, Talaromyces, Clostridium, Fusarium, Thielavia, Thermomyces, Athelia, Humicola, Penicillium, Artomyces, Gloeophyllum, Pycnoporus, Steccherinum, Trametes etc. Suitable commercially-available glucoamylases, include AMG 200L; AMG 300 L; SAN™ SUPER and AMG™ E (Novozymes); OPTIDEX® 300, OPTIDEX L-400, DISTILLASE® CX and DISTILLASE® PLUS (IFF); AMIGASE™ and AMIGASE™ PLUS (DSM); G-ZYMER G900 an G-ZYMER G990 ZR (Enzyme Bio-Systems).

Glucoamylases typically are added in an amount of about 0.1 to 2.0 glucoamylase units per gram dissolved solids (i.e., GAU/g DS), e.g., about 0.16 GAU/g DS, 0.23 GAU/g ds, 0.33 GAU/g DS and the like.

### IV. No Significant Increase in Ethyl-Glucoside

An advantage of the present methods is that they do not result in the production of significant amounts of ethyl-glucoside in fermentation substrates. The production of ethyl glucoside reflects a direct loss of product production and is not acceptable.

It has now been shown that when added prematurely in a fermentation, TGL activity indeed results in increased amounts of glyceryl-glucoside and ethyl-glucoside. The increase in these products suggests that both ethanol and glycerol, respectively, can act as an acceptor in the transglycosylation reaction. However, these undesirable products are largely avoided by the addition of an enzyme having TGL activity under the described late-fermentation conditions.

### V. Modes of Addition of Enzymes with Transglucosidase and Glucoamylase Activity

Where a fermentation substrate initially has low maltose and maltotriose content, TGL may be added from the start of fermentation with no observed adverse effects. In such fermentation substrates, the amount of short linear maltooligosaccharides is sufficiently low to avoid the formation of poorly digestible branched maltooligosaccharides that are not good substrates for glucoamylases.

Where a fermentation substrate initially has higher maltose and maltotriose content, enzymes with TGL activity are preferably added or present toward EoF, or may be added to a fermentation substrate that forms only a portion of liquefact, or a portion of final fermentation medium. For examples, TGL and GA can be added to whole-stillage including any fractionated portion of whole-stillage, where it can liberate glucose for inclusion as backset recycle in a related or unrelated fermentation substrate, including a later stage of a continuous fermentation and a subsequent or different fermentation.

### VI. Fermentation Substrates

The present methods are not limited to a particular subset of fermentation substrates, nor to a particular fermentation product or fermenting microorganism. As described, TGL liberates more glucose for fermentation, so long as it is added to or present in the fermentation at the appropriate time.

Exemplary fermentation substrates include those from dry grind fuel ethanol facilities and wet-mill carbohydrate processing facilities, and variations, thereof. Fermentation substrates also include those used for producing other valuable biochemical products, such as lactic acid and amino acids.

These and other aspects and embodiments of the present methods, and compositions resulting, therefrom, will be apparent to the skilled person in view of the present description. The following examples are intended to further illustrate, but not limit, the compositions and methods.

### EXAMPLES

The following enzymes were used in the Examples:
SPEZYME® HT: thermostable α-amylase for starch liquefaction
DISTILLASE® CX blend of Trichoderma GA, variant Aspergillus α-amylase and trehalase
DISTILLASE® PLUS blend of Trichoderma GA, Aspergillus α-amylase, trehalase and protease
DISTILLASE® PLUS WB same as above but unclarified
FERMGEN® 2.5× protease

### Example 1. Treatment of Stillage with Transglucosidase

A corn liquefact was prepared by mixing 673.1 g milled corn with 1,326.9 g water. The mixture was brought to pH 5.2 with 4 N sulfuric acid and heated to 60° C. in a water bath with continuous stirring. 134.6 μg α-amylase SPEZYME® HT was added. The mixture was maintained at 60° C. for 30 min, heated to 90° C. for 90 min and then cooled to 32° C. for use in subsequent fermentation.

2,000 g of the resulting liquefact was placed in a beaker and adjusted to pH 4.5 with 4 N sulfuric acid. 600 ppm urea, 200 ppm silicone antifoam (Sigma) and 12.3 μl FERMGEN® 2.5× were added. 100 g of this fermentation feed was placed in each of two 200 ml Erlenmeyer flasks with foam stoppers. Fermentation was initiated by adding 20.8 μl DISTILLASE® PLUS and 0.1% w/w hydrated active dry yeast (ETHANOL RED®, Fermentis, FR) to the first flask and 13.1 μl DISTILLASE® CX with the same 0.1% w/w hydrated active dry yeast to the second flask.

The flasks were maintained in a water bath at 32° C. for 66 hr. Ethanol was evaporated at 85° C. for 1.5 hr. The remaining stillage was homogenized in a blender (Braun Multiquick 5) at maximum speed for 128 sec with 1 min rest between each of 3 cycles. The pH was adjusted from 3.9 to 4.3 (using 4 N NaOH) and the pH-adjusted stillage was used for screening the effects of different enzyme activities.

Screening was performed in 96-well microtiter plates. To each well was added 0.19 ml prepared stillage with a DS of 5%. To some wells, only GA was added at a dose of 0.1 kg/MT DS. The GA used were DISTILLASE® CX or DISTILLASE® PLUS. In other wells, both the GA at a dose of 0.1 kg/MT and TRANSGLUCOSIDASE® L2000 (TGL) at a dose of 1.0 or 5.0 kg/MT was added.

The reactions were incubated for 18 hr at 32° C. in a shaker at 220 rpm. Following 18 hr the reactions were stopped by adding 0.15 ml 0.1 N sulfuric acid and glucose release was measured spectrophotometrically using a GOPOD assay. 5 μl of filtered supernatant from the quenched samples was mixed with 80 μl GOPOD reagent (Megazyme) and incubated for 20 min at 50° C. Absorbance was measured at 510 nm, where the OD corresponds to the amount of glucose released. The performance index (PI) is the amount of glucose released in the presence of TGL divided by the amount of glucose released by GA alone. When this PI is significantly above 1, the TGL releases additional glucose in the stillage compared to GA alone. Table 1, below, provides the PI numbers for the screening experiments.

TABLE 1
PI results from stillage treated with and without TGL

| Enzyme added to stillage | GA used in screening | PI |
|---|---|---|
| DISTILLASE ® PLUS | DISTILLASE ® CX | 1.7 |
| DISTILLASE ® PLUS | DISTILLASE ® CX | 1.6 |
| DISTILLASE ® PLUS | DISTILLASE ® PLUS | 2.2 |
| DISTILLASE ® PLUS | DISTILLASE ® PLUS | 1.8 |
| DISTILLASE ® CX | DISTILLASE ® CX | 1.5 |
| DISTILLASE ® CX | DISTILLASE ® CX | 1.4 |
| DISTILLASE ® CX | DISTILLASE ® PLUS | 2.03 |
| DISTILLASE ® CX | DISTILLASE ® PLUS | 1.7 |

Table 1 shows that with both the stillage samples, prepared from a fermentation with either DISTILLASE® CX or DISTILLASE® PLUS, the combination of a TRANSGLUCOSIDASE® L2000 and a glucoamylase releases significantly more glucose from stillage than glucoamylase alone. With both GA used in the screening, the combination with TGL releases at least 1.4-times more glucose. These results suggest that TGL hydrolyzes oligo- and/or polysaccharides that are not hydrolyzed by the glucoamylase during fermentation.

### Example 2. Analysis of Iso-Sugars During Fermentation

Sugar iso-forms present during fermentation were analyzed by high-performance anion-exchange chromatography using pulsed amperometric detection (HPAE-PAD), which is capable of separating isomers such as maltotriose, panose and isomaltotriose. For this analysis, 100 μl fermentation sample was taken, diluted 1,000-times, boiled for 10 min and filtered. 10 μl sample was injected on a Carbopac PA200 column (3 mm×250 mm) installed with a guard column at a flow rate of 0.5 ml/min and a temperature 30° C. PAD was performed with a cell temperature of 25° C. During the 60 min chromatographic run the following conditions were used: (i) prior to sample injection, the column was equilibrated for 10 min with 10% 1 M NaOH and 10% 500 mM NaOAc in 80% MilliQ water. The separation of the sugars was accomplished by elution with constant 10% 1 M sodium hydroxide and 90% MilliQ water for 5 minutes. During the following 5 minutes a gradient with 500 mM NaOAc was started where the % NaOAc in the mobile phase increased from 0 to 8% and % of MilliQ water decreased from 90 to 82%. In the following 50 minutes the gradient changed and % MilliQ in the mobile phase decreased from 82% to 0% and % NaOAc increased from 8 to 90%. The gradient is shown in Table 2.

TABLE 2
Chromatographic gradient

| Time (min) | Water (%) | 1M NaOH (%) | 500 mM NaAc (%) |
|---|---|---|---|
| 0-5 | 90 | 10 | |
| 5-10 | 90-82 | 10 | 0-8 |
| 10-60 | 82-0 | 10 | 8-90 |

With the appropriate calibration curve, the HPAEX-PAD chromatograms can be used to calculate the content of each of these iso-sugars in % w/v. Commercially available reference sugars are available for the glucose, fructose, isomaltose (IM2), maltulose, isomaltotriose (IM3), maltose (M2), isomaltotetraose (IM4), panose, isomaltopentaose (IM5), maltotriose (M3), isomaltohexaose (IM6), isomaltoheptaose (IM7), maltotetraose (M4), maltopentaose (M5), maltohexaose (M6) and maltoheptaose (M7).

In addition to peaks corresponding to reference sugars, the HPAEX-PAD chromatograms show other peaks, most likely corresponding to branched oligosaccharides. Because there are no commercially available reference sugars for these branched oligosaccharides, they were labeled using defined criteria. An unknown peak falling between maltotriose and maltotetraose was referred to as a branched maltotetraose (B4). The first peak that appears following maltotriose was referred to as B4-a, the second B4-b and so on. Peaks falling between maltotetraose and maltopentaose were referred to as B5-a, B5-b etc. All peak areas for branched DP4-a, b, c, d, e and so forth were added together and referred to as branched DP4. The same was done for DP5-a, b, c etc., DP6-a, b, c etc. and DP7-a, b, c etc. Given the absence of reference standards, the peak areas of the chromatograms cannot be converted to concentrations. For these peaks, the peak areas of the chromatograms (in nC·min) will be used for comparisons between fermentation time and runs.

### Example 3. Adding Transglucosidase in Fermentation

Corn liquefact was prepared using SPEZYME® HT as in Example 1. The pH was adjusted to 4.5 with sulfuric acid and 600 ppm urea, 200 ppm silicone antifoam (Sigma) and 0.02 kg/MT DS FERMGEN® 2.5× were added. 100 g liquefact were put in several 200 ml Erlenmeyer flasks for incubation with stirring in a water bath at 32° C. A water-lock was placed on top of each flask. Fermentation was started by adding 0.9 kg/MT DS DISTILLASE® PLUS WB with or without 0.01 or 0.1 kg/MT TRANSGLUCOSIDASE L2000 and 0.1% w/w hydrated active dry yeast (as before). Fermentations were performed in triplicate. Samples were taken at 72 hr fermentation and analyzed for ethanol by HPL. The averaged results are shown in Table 3.

TABLE 3
Ethanol levels during fermentation with and without TGL

| | Ethanol (%v/v) | | |
|---|---|---|---|
| Time (hr) | no TGL | 0.01 kg/MT TGL | 0.1 kg/MT TGL |
| 15 | 9.0 | 9.0 | 9.0 |
| 23 | 12.4 | 12.4 | 12.7 |
| 39.5 | 16.5 | 16.7 | 17.3 |
| 46 | 17.2 | 17.4 | 17.5 |
| 63.5 | 17.9 | 17.9 | 17.8 |
| 70 | 17.9 | 17.9 | 17.8 |

Adding TGL to fermentations resulted in faster ethanol formation as the ethanol level in the middle part of the fermentation was higher. At higher TGL doses, the rate of ethanol production was higher than at lower doses. However, at end of fermentation (EoF), the lowest dose of TGL does not yield higher ethanol production, and the highest dose appears to negatively affect ethanol levels.

### Example 4. Staged Dosing of Transglucosidase in Fermentation

As shown in Example 3, adding TGL at the beginning of fermentation can result in lower ethanol yields. TGL addition at different times after starting fermentation (i.e., 40-64 hr), was then examined. To compensate for the shorter time in which the enzyme is present in the fermentation, a higher dose was added. The experiment as described in Example 3 was repeated, with the difference that TRANSGLUCOSIDASE® L2000 was added after 40 hours of fermentation at a dose of 0.05 and 0.25 kg/MT DS or after 64 hours at a dose of 0.1 and 0.5 kg/MT DS. The results are shown in Table 4, averaged for triplicate experiments.

TABLE 4
Ethanol levels during fermentation with and
without TGL dosed later in fermentation

| | Ethanol (% v/v) | | | | |
|---|---|---|---|---|---|
| | | 0.05 kg/MT | 0.25 kg/MT | 0.1 kg/MT | 0.5 kg/MT |
| | no | TGL after | TGL after | TGL after | TGL after |
| Time (hr) | TGL | 40 hr | 40 hr | 64 hr | 64 hr |
| 15 | 9.0 | 9.0 | 9.1 | 9.1 | 9.1 |
| 23 | 12.4 | 12.4 | 12.3 | 12.5 | 12.4 |
| 39.5 | 16.5 | 16.6 | 16.5 | 16.6 | 16.5 |
| 46 | 17.2 | 17.4 | 17.4 | 17.4 | 17.3 |
| 63.5 | 17.9 | 18.0 | 18.1 | 17.9 | 17.9 |
| 70 | 17.9 | 18.0 | 18.1 | 18.0 | 18.0 |

The results show that when TGL is added later in the fermentation, higher ethanol yields are obtained than when TGL is not added in the fermentation.

### Example 5. Timing for Transglucosidase Dosing During Fermentation

As shown in the previous Examples, adding TGL early in fermentation has a negative effect on ethanol yield, while adding it later has a positive effect on ethanol yield. TGL was then added in fermentations over a wider time range to identify the best dosing point. Dosages and timings tested are summarized in Table 5. Sample analysis was as described in Examples 2 and 3. Tables 6, 7 and 8 shows the relative ethanol, DPn and DP2 values, respectively, (in percent) compared to the reference fermentation without added TGL.

TABLE 5
Dosing scheme for TRANSGLUCOSIDASE L2000 addition.

| Time of addition of TGL (hr) | TGS dose added (kg/MT) |
|---|---|
| N/A | None |
| 0 | 0.01 |
| 0 | 0.05 |
| 0 | 0.10 |
| 0 | 0.50 |
| 0 | 0.50 |
| 4 | 0.01 |
| 4 | 0.05 |
| 4 | 0.10 |
| 4 | 0.50 |
| 8 | 0.05 |
| 8 | 0.10 |
| 8 | 0.50 |
| 24 | 0.10 |
| 24 | 0.50 |
| 24 | 1.0 |
| 32 | 0.10 |
| 32 | 0.50 |
| 32 | 1.0 |
| 40 | 0.5 |
| 40 | 1.0 |
| 48 | 0.5 |
| 48 | 1.0 |
| 56 | 0.5 |
| 56 | 1.0 |
| 64 | 0.5 |
| 64 | 1.0 |

TABLE 6
Relative amounts of ethanol after 72 hr fermentation
with TGL added at different timepoints and dosing

| TGL | | | | | | | | | |
|---|---|---|---|---|---|---|---|---|---|
| dose | | | | | | | | | |
| (kg/ | Time of dosing TGL (hr) | | | | | | | | |
| MT) | 0 | 4 | 8 | 24 | 32 | 40 | 48 | 56 | 64 |
| 0.01 | 98.39 | 99.62 | | | | | | | |
| 0.05 | 98.55 | 99.14 | 98.32 | | | | | | |
| 0.10 | 97.68 | 97.91 | 97.03 | 98.59 | 99.43 | | | | |
| 0.50 | 95.41 | 96.14 | 95.28 | 98.01 | 99.15 | 99.42 | 98.91 | 100.6 | 99.94 |
| 1.0 | | | | 98.56 | 98.97 | 99.13 | 99.08 | 100.16 | 101.09 |

The data in Table 6 confirm that addition of TGL early in fermentation had a negative effect on ethanol yield. There was a clear dose response, where the effect was larger with higher TGL doses. When TGL was added before 48 hr, higher doses resulted in lower ethanol yields. Comparing the results at equal doses in view of the time of addition, it is clear that adding TGL later in fermentation resulted in higher ethanol yields. When TGL was added at 56 hr or later, there was a positive effect on ethanol yield compared to no TGL addition (i.e., relative ethanol values above 100%).

TABLE 7
Relative amounts of DPn after 72 hr fermentation
with TGL added at different timepoints and dosing

| TGL dose | Time of dosing TGL (hr) | | | | | | | | |
|---|---|---|---|---|---|---|---|---|---|
| (kg/MT) | 0 | 4 | 8 | 24 | 32 | 40 | 48 | 56 | 64 |
| 0.01 | 101.06 | 102.55 | | | | | | | |
| 0.05 | 103.48 | 104.89 | 103.02 | | | | | | |
| 0.1 | 105.9 | 106.18 | 105.69 | 103.63 | 101.71 | | | | |
| 0.5 | 114.99 | 114.39 | 111.16 | 107.79 | 106.71 | 105.33 | 103.56 | 101.11 | 101.45 |
| 1.0 | | | | 110.9 | 107.03 | 107.96 | 106.27 | 103.84 | 100.4 |

As shown in Table 7, the timing of TGL addition had a large effect on DPn (maltooligosaccharide with DP4 and higher) content at EoF. When TGL was added early in fermentation, DPn at EoF is up to 15% higher than the reference. The remaining DPn at EoF was correlated to the TGL dose and time of addition. The earlier TGL was added, the higher the DPn at EoF; and the higher the dose, the higher the DPn at EoF.

TABLE 8
Relative amounts of DP2 after 72 hr fermentation
with TGL added at different timepoints and dosing

| TGL dose | Time of dosing TGL (hr) | | | | | | | | |
|---|---|---|---|---|---|---|---|---|---|
| (kg/MT) | 0 | 4 | 8 | 24 | 32 | 40 | 48 | 56 | 64 |
| 0.01 | 122.93 | 133.48 | | | | | | | |
| 0.05 | 146.09 | 134.35 | 115.88 | | | | | | |
| 0.1 | 144.86 | 126.02 | 106.87 | 80.66 | 69.9 | | | | |
| 0.5 | 88.075 | 82.48 | 76.48 | 64.79 | 61.55 | 60.11 | 59.49 | 60.9 | 63.37 |
| 1 | | | | 61.42 | 57.85 | 57.49 | 57.45 | 58.63 | 60.76 |

Addition of TGL also resulted in changes in the content of DP2 and DP3 at EoF. Table 8 shows the relative DP2 values. At lower doses of TGL, and when TGL was added early in fermentation, DP2 was higher than the reference. At higher doses, or when added later in fermentation, the effect of TGL addition was smaller, and even reversed. Similar results were observed for DP3 but the differences with and without TGL were smaller. At doses of 0.5 kg/MT and more, the DP2 content was approximately 40-43% lower than the reference, and the DP3 content was approximately 5-7% lower.

HPAEX-PAD chromatography, described in Example 2, was used to analyze a subset of the fermentations described above (data not shown). The analysis showed no significant differences in the amounts of glucose during fermentation with or without added TGL. For the iso-sugars maltose, maltotriose and maltotetraose, analysis showed that the content of these linear sugars dropped faster when TGL was added to fermentations. At EoF, the content of these iso-sugars was the same as when no TGL was added. The faster drop in iso-sugars was consistent with TGL using these substrates as donors and/or acceptors.

Specifically, based on the analysis, it was determined that the content of isomaltose and panose initially increased when TGL was added in fermentation and dropped near EoF, (ii) there was less isomaltose remaining at EoF when TGL was added, (iii) panose content increased upon addition of TGL and dropped below detection at EoF, independent of TGL addition. Increased isomaltose and panose content indicated that the TGL used glucose and maltose as acceptor molecules. Analysis further showed that addition of TGL resulted in a greater amount of branched DP4, DP5 and DP6 oligosaccharides at EoF. These oligosaccharides were highest when TGL was added early in fermentation, and nearly the same when added after 40-48 hr. This analysis lead to the conclusion that TGL addition early in fermentation had a negative effect on ethanol yield.

Maltose and maltotriose concentrations measured in the fermentations without addition of TGL are shown in Table 9. Maltose concentrations during fermentation without TGL were 0.67, 0.033 and 0.013% w/v after 30, 40 and 48 hr fermentation, respectively. For maltotriose, the concentrations were 0.57% w/v after 8 hr and 0.044 after 24 hr. In a several cases, maltose and maltotriose were below detectable limits, indicated by a dash.

TABLE 9
Maltose and maltotriose concentrations during fermentation

| Time (hr) | Maltose (% w/v) | Maltotriose (% w/v) |
|---|---|---|
| 4 | 2.863 | 0.867 |
| 8 | 4.002 | 0.568 |
| 24 | 2.339 | 0.044 |
| 30 | 0.67 | 0.001 |
| 40 | 0.033 | — |
| 48 | 0.013 | — |
| 54 | 0.006 | — |
| 64 | 0.006 | — |
| 72 | 0.003 | — |

HPLC analysis distinguished two additional peaks in samples from fermentations using TGL. These peaks were identified as glyceryl-glucoside and ethyl-glucoside, which were produced when TGL was added, particularly at the start of fermentation. The production of these glucosides suggests that both ethanol and glycerol were acting as acceptor molecules for the transglycosylation reaction.

When TGL was added at the start of the fermentation glyceryl-glucoside was formed immediately and reached maximum around 24-40 hr. As fermentation progressed, the amount of glyceryl-glucoside decreased, and by 72 hr was nominally present. This suggested that the formed glyceryl-glucoside is hydrolyzed, presumably by one or more enzymes remaining or present later in fermentation. When TGL is added 32-40 hr after the start of fermentation, the glyceryl-glucoside content is the same as the reference fermentation performed without adding TGL.

Ethyl glucoside is also formed when TGL is added at the start of the fermentation, and continues to be formed throughout fermentation. The formation of ethyl-glucoside may explain why addition of TGL early in fermentation results in lower ethanol yields. When TGL is added 32-40 hr after the start of fermentation, little or no ethyl-glucoside was formed.

### Example 6. Addition of Transglucosylase to High Glucose Fermentations

The forgoing examples generally describe fermentation conditions as found in dry-grind, starch-consuming, ethanol-producing plants, which are well known in the industry. Other ethanol plants produce ethanol from rich-glucose streams that can be fermented, for example glucose and fructose syrups, greens and/or raffinate. Such rich fermentation feeds can be rich in sugars larger than glucose, and can contain significant amounts of isomaltose, panose and/or higher branched sugars. At the same time, they are low in linear maltose, maltotriose, maltotetraose etc.

To determine whether the addition of TGL at the start of fermentation of a glucose-rich syrup with low amounts of maltose and/or maltotriose increased fermentation, an exemplary glucose fermentation feed was obtained from a wet-mill operation plant. The substrate was a mixture of 70% glucose syrup and 30% corn steep liquor containing 14% w/v glucose, 0.8% w/v fructose, 0.9% w/v di-saccharides and 1.4% w/v oligosaccharides with DP3 or higher (DP3+). The maltose concentration in this substrate was 0.594% w/v and maltotriose was not detected. Because this fermentation substrate is low in maltose and maltotriose, it is a well-suited to assuring that adding TGL to fermentations with low amounts of maltose and maltotriose results in no undesirable effect.

This fermentation substrate had a pH of 4.0-4.5 and was not adjusted. 400 ppm silicone antifoam (Sigma) was added and 100 g fermentation feed was distributed to 200 ml Erlenmeyer flasks with foam stoppers. These flasks were brought to 32° C. in a water bath and stirred at 150 rpm. At the start of the fermentation 0.26 kg/MT DS OPTIMAX® 4060VHP was added to each flask and 0.1% w/w hydrated active dry yeast (as above). At 0, 4, 8 and 24 hr of fermentation, TRANSGLUCOSIDASE® L2000 was added to different flasks as show in Table 10. Experiments were performed in triplicate.

TABLE 10
Dosing scheme for TGL addition

| Fermentation | Time of addition of | TGS dose added |
|---|---|---|
| No. | TGL (hr) | (kg/MT) |
| 1 | — | — |
| 2 | 0 | 0.01 |
| 3 | 0 | 0.05 |
| 4 | 0 | 0.1 |
| 5 | 0 | 0.5 |
| 6 | 4 | 0.01 |
| 7 | 4 | 0.05 |
| 8 | 4 | 0.1 |
| 9 | 4 | 0.5 |
| 10 | 8 | 0.05 |
| 11 | 8 | 0.1 |
| 12 | 8 | 0.5 |
| 13 | 24 | 0.1 |
| 14 | 24 | 0.5 |
| 15 | 24 | 1.0 |

At appropriate time intervals samples were taken for HPLC analysis as described in Examples 2 and 3. For each sample, the relative difference compared to the reference fermentation (No. 1) without the additional of transglucosidase was calculated. For each sample time point, the average amount of ethanol produced was divided by the average amount produced in the reference sample (expressed in percent). A positive effect from transglucosidase was indicated by a value above 100%, and a negative effect by a value below 100%. DPn, DP3, DP2 and DP1 sugar content was analyzed in the same way. Table 11 shows the relative ethanol amounts compared to the reference fermentation without transglucosidase added.

TABLE 11
Relative amounts of ethanol after 54 h fermentation with
transglucosidase added at different timepoints and dosing

| | Time of dosing TGL (hr) | | | |
|---|---|---|---|---|
| TGL dose (kg/MT) | 0 | 4 | 8 | 24 |
| 0.01 | 100.86 | 100.25 | | |
| 0.05 | 100.51 | 100.82 | 99.92 | |
| 0.1 | 101.14 | 101.9 | 100.97 | 100.38 |
| 0.5 | 101.7 | 101.14 | 101.57 | 101.42 |
| 1 | | | | 101.78 |

As shown in Table 11, in all fermentations where TGL was added, ethanol yield at 54 hr fermentation was higher than in the reference fermentation without TGL. The relationship between TGL and relative ethanol yield was dose-dependent. There did not appear to be a correlation between the time of TGL addition and relative ethanol yield. As seen in the previous Example, addition of TGL resulted in a significant drop in DP3 and DP2 values. In contrast with the previous Example, adding TGL to this high glucose substrate also resulted in a drop in DPn values. DP2 values were up to 80% lower when TGL was added compared to the reference. DP3 values were up to 75% lower and DPn values up to 7% lower. The lower DPn, DP3 and DP2 are dependent on the TGL dose and not on the time of addition.

For one of the fermentations, where TGL was added after 24 hr, an end-of-fermentation sample was analyzed for iso-sugars as described in Example 2. The result of the analysis with HPAE-PAD, shown in Table 12, demonstrated that during fermentation of rich-glucose substrates isomaltose, maltulose and panose were degraded efficiently by the addition or TGL. TGL can therefore efficiently hydrolyse substrates that the GA alone cannot hydrolyze, with the potential for higher ethanol yields.

TABLE 12
EoF saccharide content with and without TGL

| Sugar | no TGL | with TGL |
|---|---|---|
| Trehalose | 0.006 | 0.005 |
| Glucose | 0.006 | 0.005 |
| Fructose | 0.002 | 0.002 |
| Melibiose | 0.012 | 0.013 |
| Isomaltose | 0.133 | 0.000 |
| Maltulose | 0.173 | 0.011 |
| Kojibiose | 0.007 | 0.000 |
| Gentiobiose | 0.015 | 0.018 |
| Isomaltotriose | 0.000 | 0.000 |
| Nigerose | 0.003 | 0.000 |
| Maltose | 0.001 | 0.000 |
| Panose | 0.043 | 0.000 |

### Example 7: Corn Fermentation with GA and TGL-Treated Stillage

Thin stillage (5.82% DS) from a European corn dry-grind ethanol production facility was treated with and without 0.25 kg/MT TGL+0.25 kg/MT DISTILLASE® PLUS WB.

Incubation of treated thin stillage was performed for 18 hr at 45° C., pH 4.65. Enzymes were added on a dry solids base at 5.82% DS. This treated thin stillage was then added to standard corn liquefactions, in an amount of 35% w/w. Liquefaction was performed for 2.5 hours at 84° C., 32% DS corn and pH 5.1-5.2. Following the addition of thin stillage, liquefaction DS was 34.04%. SPEZYME® HT was added at the start of liquefaction, at a dose of 0.19 kg/MT corn DS. The control was liquefact to which was added untreated thin stillage.

In the liquefaction in which the stillage was treated with GA and TGL the DPn content was 0.25% lower than the control, DP3 and DP2 hardly changed and DP1 was 0.28% higher than the control. TGL and GA hydrolyzed DPn sugars into mainly DP1.

Corn liquefacts were then adjusted to pH 4.8 with sulfuric acid. 1,000 ppm urea and 400 ppm silicone antifoam (Sigma) were added and the 100 g liquefact was transferred to 100 ml Erlenmeyer flasks for incubation at 32° C. A rubber stopper pierced with a needle was placed on top of each flask. Fermentation was initiated by adding 0.93 kg/MT DS DISTILLASE® PLUS WB and 0.1% w/w hydrated active dry yeast (as above). Fermentations were run six-fold, and samples were taken at 65 hr for analysis by HPLC as described above. The relative ethanol content in the fermentation of liquefact that contained TGL+GA-treated thin-stillage was 100.6% compared to the fermentations of liquefact that contain untreated thin-stillage (data not shown).

EOF samples were analyzed on a Dionex Carbopac PA200 column, as described in Example 2. The content of isomaltose and maltulose in the fermentations containing TGL+GA-treated thin-stillage were lower than those containing untreated thin-stillage.

The results demonstrated that treating stillage with TGL+GA before being recycled into liquefaction, could result in reduction of isomaltose and maltulose content and an increased ethanol yield.

What is claimed is:

1. A method for increasing the amount of glucose available for fermentation in a fermentation substrate, comprising; contacting a fermentation substrate with an enzyme having transglucosidase activity and an enzyme having glucoamylase activity at a time when the combined amount of maltose and maltotriose in the fermentation substrate is below a selected amount.

2. The method of claim 1, wherein the selected amount is below 0.5%, below 0.05% and preferably below 0.01% w/v.

3. The method of claim 1 or 2, wherein the fermentation substrate following the contacting with the enzyme having transglucosidase activity has reduced levels of isomaltose, panose and/or maltulose, compared to otherwise identical fermentation substrate not contacted with the enzyme having transglucosidase activity.

4. The method of any one of claims 1-3, wherein the fermentation substrate following contacting with the enzyme having transglucosidase activity does not have a significant increase in amounts of ethyl-glucoside, compared to otherwise identical fermentation substrate not contacted with the enzyme having transglucosidase activity.

5. The method of any one of claims 1-4, wherein the fermentation substrate is contacted with the enzyme having transglucosidase activity and the enzyme having glucoamylase activity simultaneously.

6. The method of any one of claims 1-4, wherein the fermentation substrate is contacted with the enzyme having transglucosidase activity and the enzyme having glucoamylase activity sequentially.

7. The method of any one of claims 1-6, wherein the enzyme having transglucosidase activity is added after the start of fermentation.

8. The method of any one of claims 1-7 wherein the transglucosidase is added 32 hours, 40 hours, 48 hours, 54 hours or 64 hours after the start of fermentation.

9. The method of any one of claims 1-8, wherein the fermentation substrate is a rich-glucose fermentation substrate.

10. The method of any one of claims 1-9, wherein the fermentation substrate is thin stillage added as backset to a liquefaction.

11. The method of any one of claims 1-10, wherein the fermentation substrate is whole stillage, of which at least a portion is added to a liquefaction.

12. The method of any one of claims 1-11, wherein the fermentation substrate is a starch liquefact.

13. The method of any one of claims 1-12, further comprising fermenting the glucose made available for fermentation into ethanol, lactic acid or amino acids.

14. The method of any one of claims 1-13, further comprising fermenting the glucose made available for fermentation using yeast and/or bacteria and a fermenting organism.

15. The method of any one of claims 1-14, wherein the amount of enzyme having transglucosidase activity contacting the fermentation substrate is at least 0.017, at least 0.085, at least 0.170, at least 0.850 or at least 1.70 transglucosidase Units per gram substrate dissolved solids.
